(19) United States
(12) Patent Application Publication
(10) Pub. No.: US 2007/0118086 A1
(43) Pub. Date: May 24, 2007

(54) Method for determining vulva irritation and sensitivity

(76) Inventors: Miranda Aref Farage, Mason, OH (US); Alexandra Katsari, Ano Ilissia (GR)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/283,174

(22) Filed: Nov. 18, 2005

(51) Int. Cl. A61F 13/15

(52) U.S. Cl. 604/359

(57) ABSTRACT

A method for selecting a group of individuals having vulva irritation. The method includes; collecting facial skin information from an individual; establishing a first group of individuals having vulva irritation; establishing a second group of individuals not having vulva irritation; and placing the individual in either said first group or said second group based upon information collected on facial skin.

### FIELD OF THE INVENTION

This invention relates to a method for determining if an individual has vulva irritation and/or sensitivity.

### BACKGROUND OF THE INVENTION

Previously, to determine if an individual had vulva irritation and/or sensitivity was a cumbersome task. First, a medical professional would need to be trained to be able to identify and classify vulva irritation. This medical professional would then examine a human female to determine whether or not the female had vulva irritation. This method was time consuming, intrusive for the female and costly.

It has been found that there is a more efficient method to identify individuals who have vulva irritation and/or sensitivity without the need of using a specially trained medical professional.

### SUMMARY OF THE INVENTION

The present invention provides a method for selecting a group of individuals having vulva irritation. The method comprises the steps of: collecting facial skin information from an individual; establishing a first group of individuals having vulva irritation; establishing a second group of individuals not having vulva irritation; and placing the individual in either said first group or said second group based upon information collected on facial skin. The facial skin information collected may be facial skin sensitivity, and erythema or skin redness.

### DETAILED DESCRIPTION OF THE INVENTION

The method of the present invention relates to the selection of individuals who have vulva irritation.

As used herein, the term “absorbent articles” refers to devices which absorb and contain body exudates, and, more specifically, refers to devices which are placed against, in proximity to or within the body of the wearer to absorb and contain the various exudates discharged from the body. The term “disposable” is used herein to describe absorbent articles which are not intended to be laundered or otherwise restored or reused as an absorbent article (i.e., they are intended to be discarded after a single use and, preferably to be recycled, composted or otherwise disposed of in an environmentally compatible manner). The term “absorbent articles” includes sanitary napkins, panty liners (or pantiliners), incontinence devices, interlabial products, tampons, diapers, and training pants.

As part of manufacturing and selling products it is a common practice to test new prototype products with consumers prior to introducing the new product into the marketplace. As part of that testing there are certain individuals who are more demanding and/or critical of the products performance than other individuals. If one can conduct some early testing with those particular individuals who are of the more critical or demanding group one can streamline the development time and effort. For example, if a product tests well with the more critical and/or demanding group of individuals it has a higher likelihood of testing well with the general population of potential consumers.

With disposable absorbent article one group of demanding individuals are those who have vulva irritation and/or sensitivity. Individuals who have vulva irritation and/or sensitivity are most likely to experience discomfort with disposable absorbent articles as their skin which contacts the disposable absorbent article is irritated and/or sensitive. Such delicate skin can be further irritated by the usage of disposable absorbent articles if the disposable absorbent articles are not properly designed to meet the needs of these individuals.

In order to save time and money in the development of new disposable absorbent articles it is advisable to first test the product with those individuals who are the most critical and/or demanding. If the product is well received by this critical and/or demanding group it will likely perform well with the more general population. However, if the product does no perform well with this critical and/or demanding group the chances of it performing well with the general population are low.

In order to get a relatively quick, low cost and low effort review of a new product, the product can be tested with this more sensitive and/or demanding group of individuals. One hurdle with this approach in the past has been the cumbersome process to identify this group of individuals. To identify individuals with vulva irritation a medically trained professional had to be used to physically examine each individual to determine whether or not the individual had vulva irritation. The medical professional had to be trained to correctly identify and classify each subject examined. This process was both time consuming and costly.

The present invention provides a method for selecting individuals or a group of individuals having vulva irritation. The method comprises the steps of: a) collecting facial skin information from an individual; b) establishing a first group of individuals having vulva irritation; c) establishing a second group of individuals having vulva irritation; and d) placing the individual in either the first group or the second group based upon the information collected on facial skin.

The step of collecting the facial skin information is gathered from the individual through a question or a questionnaire. The questionnaire can be administered through the use of a computer, through an interview process or by having an individual filling out a form. The method or technique used to gather the information can be carried out in any way known to gather information from individuals.

The following are examples of suitable questions given to an individual.

1. Is your facial skin sensitive?
a. Non sensitive
b. Somewhat sensitive
c. Sensitive
d. Very sensitive

2. Do you have redness on your facial skin?
a. No redness
b. Slight redness
c. Moderate redness
d. Severe redness

With respect to question 1 if the individual answers positive to any one of b. Somewhat sensitive, c. Sensitive, or d. Very sensitive, the individual will be placed in the group having vulva irritation. If the individual answers positive to a. Non sensitive, the individual will be placed in the group not having vulva irritation. The questionnaire can be given to any size group of individuals.

With respect to question 2 if the individual answers positive to any one of b. Slight redness, c. Moderate redness, or d. Severe redness, the individual will be placed in the group having vulva irritation. If the individual answers positive to a. No redness, the individual will be placed in the group not having vulva irritation. The questionnaire can be given to any size group of individuals.

Questions 1 and 2 can be given independently or in combination as part of the facial skin information collection process.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for selecting a group of individuals having vulva irritation, said method comprising the steps of
collecting facial skin information from an individual;
establishing a first group of individuals having vulva irritation;
establishing a second group of individuals not having vulva irritation; and
placing the individual in either said first group or said second group based upon information collected on facial skin sensitivity.

2. The method of claim 1 wherein the facial skin information is facial skin sensitivity.

3. The method of claim 1 wherein the facial skin information is facial skin redness.
